(19) United States
(12) Patent Application Publication
(10) Pub. No.: US 2019/0022210 A1
(43) Pub. Date: Jan. 24, 2019

(54) NOVEL PLATFORM DNA VACCINE

(71) Applicants: Victor G. Solodushko, Mobile, AL (US); Brian Fouty, Mobile, AL (US); Vira Bitko, Mobile, AL (US)

(72) Inventors: Victor G. Solodushko, Mobile, AL (US); Brian Fouty, Mobile, AL (US); Vira Bitko, Mobile, AL (US)

(21) Appl. No.: 15/774,178

(22) PCT Filed: Nov. 4, 2016
(86) PCT No.: PCT/US16/60631

§ 371 Date: May 7, 2018

Related U.S. Application Data
(60) Provisional application No. 62/285,732, filed on Nov. 6, 2015.
(60) Provisional application No. 62/417,663, filed on Nov. 4, 2016.

(51) Int. Cl. A61K 39/155; A61P 31/14; C07K 14/005; A61K 39/12

(52) U.S. Cl. CPC A61K 39/155; A61P 31/14; A61K 2039/53; A61K 39/12; C07K 14/005

(57) ABSTRACT

The present invention relates to novel DNA vaccines, configured to induce a robust and sustained immune response, and methods of use thereof. DNA vaccines proposed herein are configured to achieve this immune response by fusing the extracellular domain of a viral fusion protein to selected antigens or antigen-binding polypeptides. After the expression and secretion of the viral fusion-antigen protein from the initially transfected cells, the natural ability of the viral fusion protein to fuse to cell membranes and actively enter cells will allow for passive delivery of the fused target epitopes into neighboring cells, thus inducing a more robust immune response. The presented method described herein allows for the use of this DNA vaccine against known antigens present in proteins produced by infectious agents or cancer cells within a subject, against unknown antigens produced by infectious agents or cancer cells within a subject, or against naturally-occurring or synthetically-derived antigens delivered by other routes, such as injection.

### CROSS-REFERENCE TO RELATED APPLICATION

This is the national stage application of and claims priority to International Application No. PCT/US2016/060631, entitled “Novel Platform DNA Vaccine” and having an international filing date of Nov. 4, 2016, which is incorporated herein by reference. International Application No. PCT/US2016/060631 claims priority to U.S. Provisional Patent Application No. 62/285,732, entitled “Novel Platform DNA Vaccine” and filed on Nov. 6, 2015, and U.S. Provisional Patent Application No. 62/417,663, entitled “Novel Platform DNA Vaccine” and filed on Nov. 4, 2016, each of which is incorporated herein by reference.

### BACKGROUND OF THE INVENTION

Communicable diseases and cancer represent a worldwide health problem making their prevention and treatment a public health priority. Vaccines have eliminated naturally occurring cases of smallpox, have nearly eliminated polio, and have reduced the incidence and severity of numerous diseases, such as typhus, rotavirus, hepatitis A, and hepatitis B. Despite these successes, no effective vaccines currently exist to address other diseases and conditions, such as cancer, AIDS, hepatitis C, malaria, and tuberculosis, which collectively kill millions of people worldwide each year.

Deoxyribonucleic acid (DNA) vaccination utilizes genetically engineered DNA that encodes for specific antigens, such as pathogen-specific antigens, to produce an immunologic response to such antigens in a recipient. Introduction of a DNA vaccine into a cell induces the cell to transcribe and translate the proteins encoded by the vaccine. These translated proteins are then processed and presented on the surface of these cells on a major histocompatibility complex (MHC) class I molecule. As a result, vaccination by an antigen-encoding DNA plasmid can induce humoral and cellular immune responses against cancer, pathogenic parasites, bacteria, and viruses that express the selected antigen.

Unfortunately, the efficacy of DNA vaccines in clinical trials has been disappointing. Indeed, only four DNA vaccines are currently approved for use in animals, and none are approved for use in humans. The major limitation of DNA vaccines has been their inability to generate a strong humoral (antibody) and/or T cell-mediated (CD4+ helper T cell and/or CD8+ cytotoxic T cell) immune response and the inability of transcribed product to be secreted from antigen-producing transfected cells. Herein, the Applicants describe novel compositions and methods of use that induce a more robust immune response by increasing the duration and the level of antigen expression, which cannot be efficiently accomplished by current DNA vaccines.

### SUMMARY OF INVENTION

In certain embodiments, the present disclosure pertains to a DNA vaccine composition comprising: a DNA vector containing at least one isolated nucleotide sequence; wherein each nucleotide sequence encodes a multi-domain protein conjugate comprising at least one extracellular domain of a fusion protein from an enveloped virus and at least one additional domain. In some embodiments, the DNA vector is configured to integrate stably into the genome of a target cell. In some embodiments, the DNA vector is configured to transiently express the at least one antigen in a target cell. In some embodiments, the fusion protein is modified. In some embodiments, the fusion protein is truncated. In some embodiments, the fusion protein is a protein expressed by the Paramyxoviridae family. In some embodiments, the fusion protein is Respiratory Syncytial Virus F protein (RSV-F). In some embodiments, the at least one additional domain comprises an antigen. In some embodiments, the antigen is selected from the group consisting of parasite, viral, bacterial, fungal, and cancer cell antigens. In some embodiments, the antigen is fused directly to the enveloped fusion protein. In some embodiments, the antigen is fused directly to another protein that interacts with the enveloped fusion protein. In some embodiments, the antigen is not directly fused to the enveloped fusion protein. In some embodiments, the antigen interacts with the enveloped fusion protein via a polypeptide connected by a covalent or non-covalent bond. In some embodiments, the at least one additional domain comprises an antigen-binding polypeptide configured to interact with a previously-delivered or naturally occurring antigen.

In other embodiments, the present disclosure pertains to a method of eliciting an immune response against an antigen in a subject, comprising the steps of: administering a DNA vaccine to a subject; wherein the DNA vaccine comprises a DNA vector containing at least one isolated nucleotide sequence; wherein each nucleotide sequence encodes a multi-domain protein conjugate comprising at least one fusion protein from an enveloped virus and at least one additional domain. In some embodiments, the DNA vector is configured to integrate stably into the genome of a target cell in the subject. In some embodiments, the DNA vector is configured to transiently express antigen in a target cell in the subject. In some embodiments, the fusion protein is from the Paramyxoviridae family. In some embodiments, the fusion protein is RSV-F protein. In some embodiments, the at least one additional domain comprises an antigen. In some embodiments, the antigen is selected from the group consisting of parasite, viral, bacterial, fungal, and cancer cell antigens. In some embodiments, the at least one additional domain comprises an antigen-binding polypeptide configured to interact with a previously-delivered or naturally occurring antigen. In some embodiments, the DNA vaccine is administered in an amount sufficient to elicit an immune response in the subject. In some embodiments, the immune response is a cytotoxic immune response. In some embodiments, the immune response is a humoral immune response. In some embodiments, the immune response includes protective immunity against the antigen.

In others embodiments, the present disclosure pertains to a method of manufacturing a medicament for use in eliciting an immune response against an antigen in a subject, comprising the step of forming a medicament comprising a DNA vaccine comprising a DNA vector containing at least one isolated nucleotide sequence, wherein each nucleotide sequence encodes a multi-domain protein conjugate comprising at least one fusion protein from an enveloped virus and at least one additional domain. In some embodiments, the DNA vector is configured to integrate stably into the genome of a target cell. In some embodiments, the DNA vector is configured to transiently express antigen in a target cell. In another embodiment, the fusion protein is from the Paramyxoviridae family. In another embodiment, the fusion protein is an RSV-F protein. In some embodiments, the at least one additional domain comprises an antigen. In some embodiments, the antigen is selected from the group consisting of parasite, virus, bacteria, fungi, or cancer cell antigens. In some embodiments, the antigen is fused directly to the fusion protein. In another embodiment, the antigen interacts indirectly with the fusion protein through another protein. In some embodiments, the at least one additional domain comprises an antigen-binding polypeptide configured to interact with a previously-delivered or naturally occurring antigen.

### BRIEF DESCRIPTION OF THE DRAWINGS

The disclosure can be better understood with reference to the following figures.

FIG. 1 illustrates evidence that RSV-F covalently linked to mCherry can transfer the fluorescent protein between cells. To demonstrate that RSV-F can transfer proteins between cells, the Applicants constructed an RSV-F-mCherry fusion protein and stably integrated it into CT26.WT cells. Cells stably expressing mCherry without RSV-F were used for the signal comparison and cells not expressing mCherry were used as a background control. Each cell population was then co-cultured with CT26.WT cells expressing eGFP for easy identification of the acceptor cells. Forty-eight hours later, cells were analyzed by flow cytometry. Cells originally expressing only eGFP accumulated mCherry when co-cultured with cells expressing mCherry fused to RSV-F (FIG. 1C) whereas there was no transfer of intracellular mCherry between cells in the absence of RSV-F (FIG. 1B). FIG. 1A shows a background signal of eGFP-expressing cells on the mCherry panel. (Only populations of eGFP expressing cells are gated, enlarged and presented in the figure.) The data indicate that RSV-F can shepherd mCherry between cells.

FIG. 2 provides an example of a three-domain coding sequence for a DNA vaccine that will lead to the expression of a fusion protein (collectively, SEQ ID NO: 1). The RSV-F protein (plain text; SEQ ID NO: 2) is linked to two antigens normally expressed on the surface of Plasmodium falciparum, Thrombospondin-Related Anonymous Protein, also known as Thrombospondin-Related Adhesive Protein (TRAP, underlined; SEQ ID NO: 3) and circumsporozoite (CS, bold and italicized; SEQ ID NO: 4) protein. The RSV-F protein will allow entry of the fusion protein (RSVF-TRAP-CS) into neighboring cells. This will multiply the number of cells that are presenting these two antigens on MHC-Class I molecules. This should increase the intensity of the cytotoxic response compared to the same DNA vaccine which lacks the RSV-F domain (TRAP-CS). The target antigens in this vaccine, the Plasmodium falciparum antigens (TRAP and CS), can be easily exchanged for other antigens to generate vaccines designed for the prevention/treatment of other diseases.

FIG. 3 shows an example of the humoral immune response induced by the proposed DNA fusion vaccine. The Applicants generated different DNA vaccine constructs that expressed two surface antigens of P. falciparum (TRAP and CS) fused together. One construct expressed a fully secretable TRAP-CS (i.e., the antigen can be both expressed and secreted by the cell); one construct expressed a TRAP-CS variation that remained attached to the extracellular surface of the membrane after secretion; and one construct (the experimental vaccine) expressed a secretable TRAP-CS that was also fused to RSV-F (RSVF-TRAP-CS; the full sequence of this vaccine is shown in FIG. 2). BALB/c mice were immunized with one of these vaccines; non-immunized age-matched animals were also included for study. To assay for antibody production, serum samples were collected three months after vaccination and incubated with CT26.WT cells that expressed the membrane-associated TRAP-CS (without RSV-F) and intracellular mCherry. Cells were then probed with FITC-labeled anti-mouse IgG. The serum from mice immunized with the experimental (RSVF-TRAP-SC) vaccine demonstrated significantly higher FITC intensity than serum from mice immunized with vaccines lacking the RSV-F domain, a result that suggested the presence of a significantly higher level of circulating specific antibodies against cells expressing TRAP and CS in RSVF-TRAP-CS-immunized mice.

FIG. 4 shows an example of a T cell immune response to the proposed DNA fusion vaccination. The Applicants generated different DNA vaccine constructs that expressed two surface antigens of P. falciparum (TRAP and CS) fused together. One construct expressed a fully secretable TRAP-CS (i.e., the antigen can be both expressed and secreted by the cell); one construct expressed a TRAP-CS variation that remained attached to the extracellular surface of the membrane after secretion; and one construct (the experimental vaccine) expressed a secretable TRAP-CS also fused to RSV-F (RSVF-TRAP-CS). A vaccine composed of RSV-F alone (i.e., without TRAP or CS) was used as a control for this experiment. BALB/c mice were immunized with one of these vaccines. Four months after vaccination, animals were stimulated by CT26.WT cells stably expressing TRAP and CS (i.e. a model of malaria-infected cells) intraperitoneally. Five days later, freshly isolated splenocytes from immunized animals were evaluated for T cell activation by analyzing cells for the expression of a surface marker of activation (CD44). Both CD4+ (FIG. 4A) and CD8+ (FIG. 4B) T cells isolated from RSVF-TRAP-CS immunized mice showed greater T cell activation following stimulation when compared to cells obtained from animals immunized with vaccines lacking the RSV-F domain or cells obtained from animals immunized with a vaccine coding RSV-F alone. This result is consistent with the presence of significantly higher levels of TRAP/CS specific effector and memory T cells in RSVF-TRAP-CS-immunized mice.

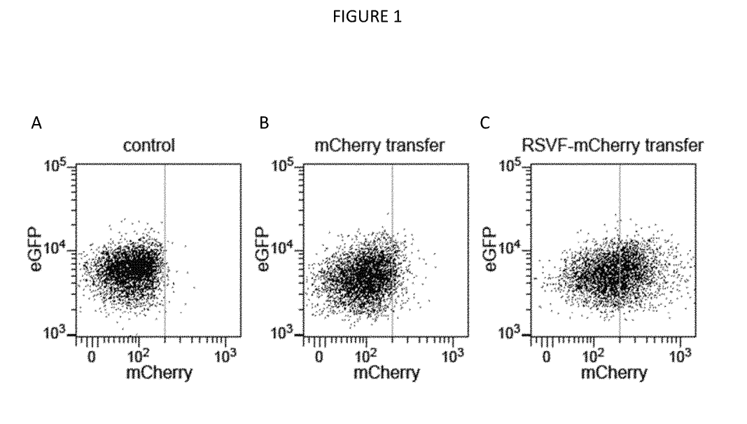

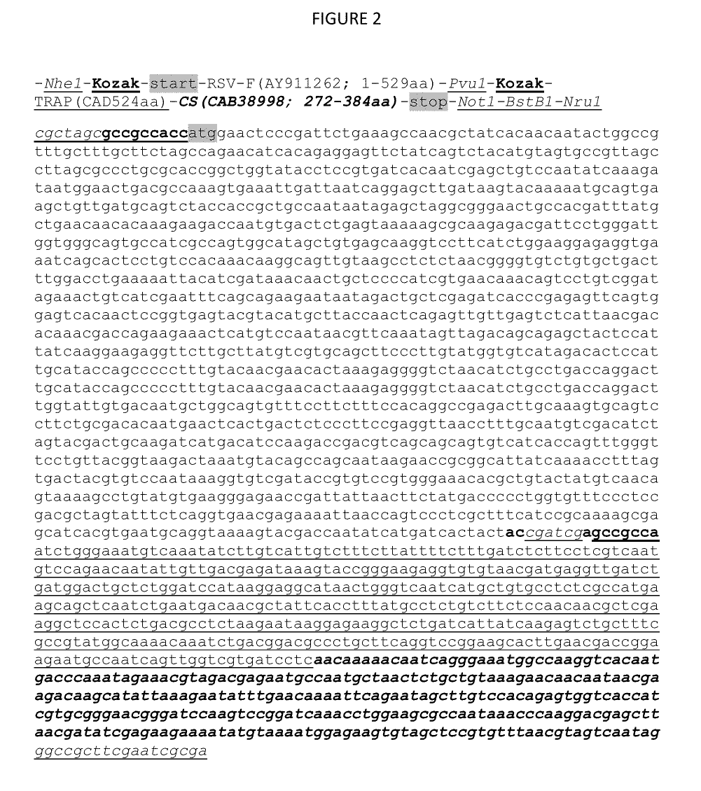

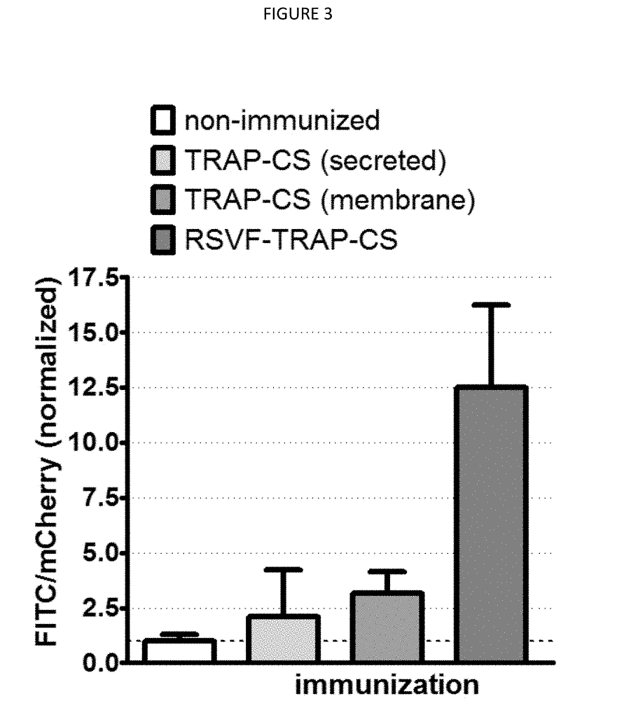

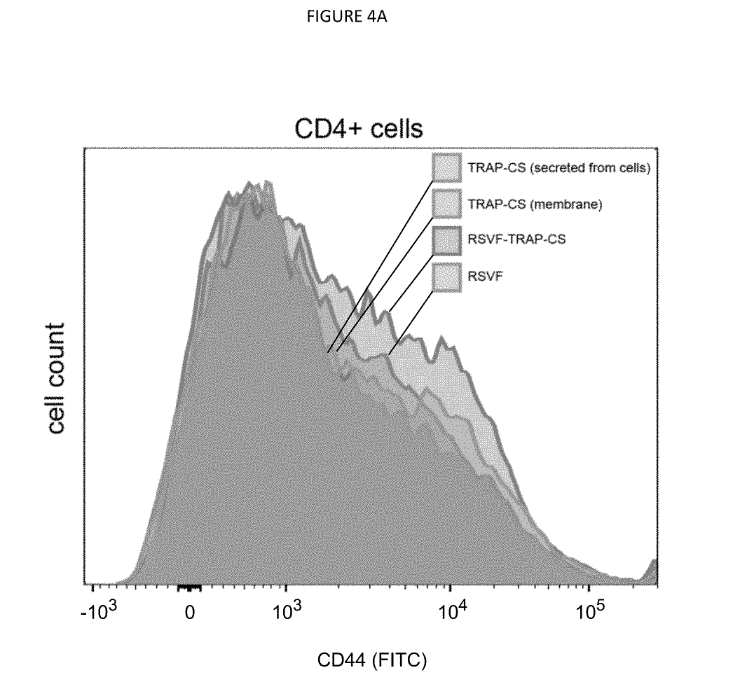

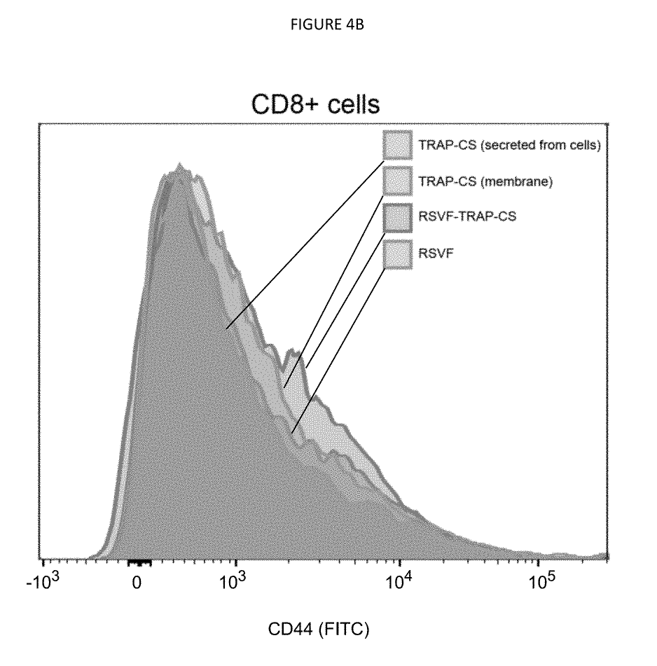

### DESCRIPTION OF THE PREFERRED EMBODIMENT

The present disclosure generally pertains to DNA vaccine compositions and methods for use thereof. In some embodiments, the DNA vaccine comprises the extracellular domain of a fusion protein from an enveloped virus cloned into a DNA vector and at least one additional domain. The additional domain may comprise an antigen from a virus, bacteria, parasite, fungi, or cancer cell or polypeptide binding the antigen indirectly. The additional domain may also comprise an antigen-binding polypeptide configured to interact with a previously-delivered or naturally occurring antigen. In some embodiments, the method comprises the step of delivering a DNA vaccine to cells in a subject via intramuscular, intraperitoneal, intravenous, or subcutaneous injection, or via inhalation or ingestion.

As used herein, “antigen” means a peptide, polypeptide or protein expressed by a virus, bacteria, parasite, fungi, or cancer cell.

As used herein, “enveloped fusion protein” means a fusion protein from an enveloped virus.

As used herein, “immune response” means a change in the phenotype of a subject's immune system. For example, an immune response may be an increase in the absolute or relative number of a particular lymphocyte subset, such as an increase in the percentage of circulating CD8+ T cells. An immune response can be measured using methods known in the art, such as flow cytometry to assess changes in the surface markers of lymphocytes from a subject.

As used herein, “modified fusion protein” means a fusion protein modified from its native state. For example, a modified fusion protein may include, but is not limited to, a protein in which one or more peptides have been altered from their native state and a native protein to which one or more additional molecules (e.g., glycosylation) or peptides have been added.

As used herein, “native fusion protein” means a fusion protein that has not been modified or truncated.

As used herein, “protective immunity” means an immune response that prevents, retards the development of, or reduces the severity of a disease, symptoms thereof, or other deleterious condition that is associated, directly or indirectly, with an antigen.

As used herein, “subject” means a vertebrate, preferably a mammal, including, but not limited to, a human.

As used herein, “target cell” means a cell to which a DNA vaccine is delivered, either in vitro or in vivo, for example within a subject.

As used herein, “truncated fusion protein” means a fusion protein in which a portion of the native fusion protein has been removed. For example, a native fusion protein may be enzymatically cleaved to remove a portion of the protein.

In certain embodiments, the present disclosure pertains to a DNA vaccine composition comprising a DNA vector containing at least one isolated nucleotide sequence encoding a multi-domain protein conjugate comprising at least one fusion protein from an enveloped virus and at least one additional domain. The additional domain may comprise at least one antigen and/or antigen-binding polypeptide. The antigen-binding polypeptide may be configured to bind to antigen that is naturally-occurring in a target cell or that was delivered to the target cell. As known in the art, the DNA vector may be configured: to integrate stably into the genome of the target cell; to stably express protein conjugate without integration into the target cell genome (for example via Adeno-Associated Virus (AAV) delivery); or to transiently express protein conjugate in a target cell.

The fusion protein in the DNA vaccine composition can be selected from different families of enveloped viruses, including, but not limited to, the RSV-F protein of the Paramyxoviridae family, HA protein of the Orthomyxoviridae family, Env protein of the Retroviridae family, S protein of the Coronaviridae family, GP protein of the Filoviridae family, GP, SSP proteins of the Arenaviridae, the E1/E2 of the Togaviridae family, E(TBEV), (E1/E2 (HPV) proteins of the Flaviviridae family, G_{N}/G_{C} proteins of the Bunyaviridae family, of the G protein of the Rhabdoviridae family, gB, gD, gH/L proteins of the Herpesviridae family, eight proteins of the Poxviridae family, and S, L proteins of the Hepadnaviridae family. The fusion protein can be a native fusion protein, a modified fusion protein, or a truncated fusion protein.

Antigens may be selected from a variety of sources. Viruses that contain antigens suitable for use in the present invention include, but are not limited to Human Immunodeficiency Virus (HIV) and Respiratory Syncytial Virus (RSV). Bacteria that contain antigens suitable for use in the present invention include, but are not limited to, organisms causing the Mycobacterium tuberculosis complex in humans (M. tuberculosis, M. bovis, M. africanum, M. microti, M. canetti, and M. pinnipedii). Fungi that contain antigens suitable for use in the present invention include, but are not limited to, Cryptococcus neoformans, Coccidioidomycosis, Blastomycosis, and Histoplasmosis. Cancer cells that contain antigens suitable for use in the present invention include, but are not limited to, adenocarcinoma, small cell, and squamous cell cancer.

In certain embodiments, the present disclosure pertains to method of administering a DNA vaccine to a subject to induce an immune response to an antigen. The DNA vaccine comprises a DNA vector containing at least one isolated nucleotide sequence encoding a multi-domain protein conjugate comprising at least one fusion protein from an enveloped virus and at least one antigen or antigen-binding polypeptide. The vector can be configured either to integrate stably in the genome of a target cell to express antigen or, alternatively, to transiently express protein conjugate. Suitable vectors are described below. The fusion protein can be from any of the aforementioned proteins. Examples of suitable parasites, viruses, bacteria, fungi or cancer cells as sources of antigens are described hereinabove. The DNA vaccine can be administered to a subject in an amount sufficient to elicit an immune response. The immune response may be cytotoxic and/or humoral. The immune response may also induce protective immunity to one or more antigens in a subject.

The DNA vaccine can be administered to a subject in an amount sufficient to induce an immune response. The immune response may be humoral and/or cellular and may induce protective immunity. Suitable routes of administering the DNA vaccine include, but are not limited to, inhalation, ingestion and intravenous, intramuscular, intraperitoneal, intradermal, and subcutaneous injection.

To prolong antigen presentation by target cells, the DNA vaccine can be delivered using a vector that has the ability to stably integrate into the genome of target cells. Suitable vectors include, but are not limited to, lentiviruses, gamma-retroviruses, and transposons.

The present disclosure also pertains to a method of manufacturing a medicament configured to induce an immune response against an antigen in a subject, the method comprising the step of forming a medicament comprising a DNA vaccine. The DNA vaccines described herein are suitable for use in this manufacturing method.

After delivery of DNA vector to target cells, the target cells express and secrete the protein conjugate. The natural ability of the viral fusion proteins to fuse with cell membranes and actively enter cells allows for delivery of the antigens into neighboring cells. This leads to the presentation of the delivered antigens on a major histocompatibility complex (MEW) class I molecule in a much larger population of cells than is possible with the initial DNA vector, thus inducing a more robust (e.g., cytotoxic) response. The fusion of antigen to the extracellular domain of a viral envelope protein also allows for antigen secretion and induction of a humoral immune response to the antigen. This innovative approach can be used to target infections in both humans and animals that are otherwise difficult or impossible to vaccinate using conventional methods.

### Examples

One specific example of how this DNA vaccine can significantly increase the number of antigen-presenting cells is to clone the coding region for the RSV-F protein along with the extracellular domains of two target antigens on the surface of Plasmodium falciparum—Circumsporozoite and Thromobospondin Related Adhesive Protein—and use this DNA vaccine for immunization. After the initially transfected cells express and secrete the RSV-F fusion protein, the natural ability of RSV-F to enter cells allows for delivery of the target antigens into neighboring, non-transfected cells. This secondary ‘infection’ results in the presentation of the delivered fusion antigens on MHC class I molecules in a much larger population of cells than is possible with the initial DNA transfection, thus inducing a more robust (e.g., cytotoxic) response. Fusion of the antigen to the extracellular domain of the viral envelope protein allows secretion of the antigen from the cell in which it was transcribed, thereby allowing a humoral response to the antigen.

### Potential Uses for the DNA Vaccine

The innovative strategy of fusing the extracellular domain of a viral envelope protein to target antigens, and to deliver them using a DNA vector, forms the basis for a platform DNA vaccine in which antigens of interest can be easily modified or replaced to induce the immune response of a subject to target different diseases. By cloning specific antigen coding regions into the platform DNA vaccine, different cancers and diseases, such as malaria, tuberculosis, melioidosis, and Dengue fever can be targeted.

The compositions and methods disclosed herein for DNA vaccines provide significant benefits compared with conventional vaccination methods. The compositions and methods enable significant increases in the presentation of antigen; further, the DNA vaccination described herein was shown to significantly increase CD8+ lymphocytes and decrease CD62L+ cells relative to control (indicative of an augmented cytotoxic response). Given the potential public health benefit afforded by an effective vaccination composition and methodology, the presently disclosed compositions and methods may result in significant clinical benefits to subjects with a variety of diseases.

This application references various publications. The disclosures of these publications, in their entireties, are hereby incorporated by reference into this application to describe more fully the state of the art to which this application pertains. The references disclosed are also individually and specifically incorporated herein by reference for material contained within them that is discussed in the sentence in which the reference is relied on.

The methodologies and the various embodiments thereof described herein are exemplary. Various other embodiments of the methodologies described herein are possible.

Now, therefore, the following is claimed:

1. A DNA vaccine composition comprising:
a DNA vector containing at least one isolated nucleotide sequence;
wherein each nucleotide sequence encodes a multi-domain protein conjugate comprising an enveloped fusion protein and at least one additional domain.

2. The composition of claim 1, wherein the at least one additional domain comprises an antigen.

3. The composition of claim 1, wherein the DNA vector is configured to integrate stably into the genome of a target cell.

4. The composition of claim 1, wherein the DNA vector is configured to transiently express the protein conjugate in a target cell.

5. The composition of claim 1, wherein the fusion protein comprises an extracellular domain of an enveloped fusion protein.

6. The composition of claim 1, wherein the fusion protein comprises a modified fusion protein.

7. The composition of claim 1, wherein the fusion protein comprises a truncated fusion protein.

8. The composition of claim 1, wherein the fusion protein is selected from the group consisting of RSV-F protein and proteins expressed by the Paramyxoviridae family.

9. The composition of claim 2, wherein the antigen is fused directly to the enveloped fusion protein.

10. The composition of claim 2, wherein the antigen is fused directly to another protein that interacts with the enveloped fusion protein.

11. The composition of claim 2, wherein the antigen is not directly fused to the enveloped fusion protein.

12. The composition of claim 1, wherein the at least one additional domain comprises an antigen-binding polypeptide.

13. A method of eliciting an immune response against an antigen in a subject, comprising the steps of:
administering a DNA vaccine to a subject in an amount sufficient to elicit an immune response in the subject;
wherein the DNA vaccine comprises a DNA vector containing at least one isolated nucleotide sequence; and
wherein each nucleotide sequence encodes a multi-domain protein conjugate comprising at least one enveloped fusion protein and at least one additional domain.

14. The method of claim 13, wherein the DNA vector is configured to integrate stably into the genome of one or more target cells in the subject.

15. The method of claim 13, wherein the DNA vector is configured to transiently express the protein conjugate in one or more target cells in the subject.

16. The method of claim 13, wherein the fusion protein is selected from the group consisting of RSV-F protein and proteins from the Paramyxoviridae family.

17. The method of claim 13, wherein the at least one additional domain comprises an antigen.

18. The method of claim 13, where in the at least one additional domain comprises an antigen-binding polypeptide.

19. The method of claim 13, wherein the immune response is a cytotoxic immune response.

20. The method of claim 13, wherein the immune response is a humoral immune response.

21. The method of claim 13, wherein the immune response includes protective immunity against the at least one antigen.

22. A method of manufacturing a medicament for use in eliciting an immune response against an antigen in a subject, comprising the step of forming a medicament comprising a DNA vaccine comprising a DNA vector containing at least one isolated nucleotide sequence, wherein each nucleotide sequence encodes a multi-domain protein conjugate comprising at least one enveloped fusion protein and at least one additional domain.

23. The method of claim 22, wherein the DNA vector is configured to integrate stably into the genome of one or more target cells.

24. The method of claim 22, wherein the DNA vector is configured to transiently express the protein conjugate in one or more target cells.

25. The method of claim 22, wherein the fusion protein is selected from the group consisting of RSV-F protein and proteins from the Paramyxoviridae family.

26. The method of claim 22, wherein the at least one additional domain comprises an antigen.

27. The method of claim 22, wherein the at least one additional domain comprises an antigen-binding polypeptide.


SEQUENCE LISTING (4 sequences)

SEQ ID NO: 1 - DNA, 2228 bp, Homo sapiens
cgctagcgccgccaccatggaactcccgattctgaaagccaacgctatcacaacaatactggccgtttgctttgcttctagccagaacatcacagaggagttctatcagtctacatgtagtgccgttagccttagcgccctgcgcaccggctggtatacctccgtgatcacaatcgagctgtccaatatcaaagataatggaactgacgccaaagtgaaattgattaatcaggagcttgataagtacaaaaatgcagtgaagctgttgatgcagtctaccaccgctgccaataatagagctaggcgggaactgccacgatttatgctgaacaacacaaagaagaccaatgtgactctgagtaaaaagcgcaagagacgattcctgggattggtgggcagtgccatcgccagtggcatagctgtgagcaaggtccttcatctggaaggagaggtgaaatcagcactcctgtccacaaacaaggcagttgtaagcctctctaacggggtgtctgtgctgactttggacctgaaaaattacatcgataaacaactgctccccatcgtgaacaaacagtcctgtcggatagaaactgtcatcgaatttcagcagaagaataatagactgctcgagatcacccgagagttcagtggagtcacaactccggtgagtacgtacatgcttaccaactcagagttgttgagtctcattaacgacacaaacgaccagaagaaactcatgtccaataacgttcaaatagttagacagcagagctactccattatcaaggaagaggttcttgcttatgtcgtgcagcttcccttgtatggtgtcatagacactccattgcataccagccccctttgtacaacgaacactaaagaggggtctaacatctgcctgaccaggacttgcataccagccccctttgtacaacgaacactaaagaggggtctaacatctgcctgaccaggacttggtattgtgacaatgctggcagtgtttccttctttccacaggccgagacttgcaaagtgcagtccttctgcgacacaatgaactcactgactctcccttccgaggttaacctttgcaatgtcgacatctagtacgactgcaagatcatgacatccaagaccgacgtcagcagcagtgtcatcaccagtttgggttcctgttacggtaagactaaatgtacagccagcaataagaaccgcggcattatcaaaacctttagtgactacgtgtccaataaaggtgtcgataccgtgtccgtgggaaacacgctgtactatgtcaacagtaaaagcctgtatgtgaagggagaaccgattattaacttctatgaccccctggtgtttccctccgacgctagtatttctcaggtgaacgagaaaattaaccagtccctcgctttcatccgcaaaagcgagcatcacgtgaatgcaggtaaaagtacgaccaatatcatgatcactactaccgatcgagccgccaatctgggaaatgtcaaatatcttgtcattgtctttcttattttctttgatctcttcctcgtcaatgtccagaacaatattgttgacgagataaagtaccgggaagaggtgtgtaacgatgaggttgatctgatggactgctctggatccataaggaggcataactgggtcaatcatgctgtgcctctcgccatgaagcagctcaatctgaatgacaacgctattcacctttatgcctctgtcttctccaacaacgctcgaaggctccactctgacgcctctaagaataaggagaaggctctgatcattatcaagagtctgctttcgccgtatggcaaaacaaatctgacggacgccctgcttcaggtccggaagcacttgaacgaccggaagaatgccaatcagttggtcgtgatcctcaacaaaaacaatcagggaaatggccaaggtcacaatgacccaaatagaaacgtagacgagaatgccaatgctaactctgctgtaaagaacaacaataacgaagacaagcatattaaagaatatttgaacaaaattcagaatagcttgtccacagagtggtcaccatcgtgcgggaacgggatccaagtccggatcaaacctggaagcgccaataaacccaaggacgagcttaacgatatcgagaagaaaatatgtaaaatggagaagtgtagctccgtgtttaacgtagtcaatagggccgcttcgaatcgcga

SEQ ID NO: 2 - DNA, 1460 bp, Homo sapiens
gaactcccgattctgaaagccaacgctatcacaacaatactggccgtttgctttgcttctagccagaacatcacagaggagttctatcagtctacatgtagtgccgttagccttagcgccctgcgcaccggctggtatacctccgtgatcacaatcgagctgtccaatatcaaagataatggaactgacgccaaagtgaaattgattaatcaggagcttgataagtacaaaaatgcagtgaagctgttgatgcagtctaccaccgctgccaataatagagctaggcgggaactgccacgatttatgctgaacaacacaaagaagaccaatgtgactctgagtaaaaagcgcaagagacgattcctgggattggtgggcagtgccatcgccagtggcatagctgtgagcaaggtccttcatctggaaggagaggtgaaatcagcactcctgtccacaaacaaggcagttgtaagcctctctaacggggtgtctgtgctgactttggacctgaaaaattacatcgataaacaactgctccccatcgtgaacaaacagtcctgtcggatagaaactgtcatcgaatttcagcagaagaataatagactgctcgagatcacccgagagttcagtggagtcacaactccggtgagtacgtacatgcttaccaactcagagttgttgagtctcattaacgacacaaacgaccagaagaaactcatgtccaataacgttcaaatagttagacagcagagctactccattatcaaggaagaggttcttgcttatgtcgtgcagcttcccttgtatggtgtcatagacactccattgcataccagccccctttgtacaacgaacactaaagaggggtctaacatctgcctgaccaggacttgcataccagccccctttgtacaacgaacactaaagaggggtctaacatctgcctgaccaggacttggtattgtgacaatgctggcagtgtttccttctttccacaggccgagacttgcaaagtgcagtccttctgcgacacaatgaactcactgactctcccttccgaggttaacctttgcaatgtcgacatctagtacgactgcaagatcatgacatccaagaccgacgtcagcagcagtgtcatcaccagtttgggttcctgttacggtaagactaaatgtacagccagcaataagaaccgcggcattatcaaaacctttagtgactacgtgtccaataaaggtgtcgataccgtgtccgtgggaaacacgctgtactatgtcaacagtaaaagcctgtatgtgaagggagaaccgattattaacttctatgaccccctggtgtttccctccgacgctagtatttctcaggtgaacgagaaaattaaccagtccctcgctttcatccgcaaaagcgagcatcacgtgaatgcaggtaaaagtacgaccaatatcatgatcactact

SEQ ID NO: 3 - DNA, 419 bp, Homo sapiens
atctgggaaatgtcaaatatcttgtcattgtctttcttattttctttgatctcttcctcgtcaatgtccagaacaatattgttgacgagataaagtaccgggaagaggtgtgtaacgatgaggttgatctgatggactgctctggatccataaggaggcataactgggtcaatcatgctgtgcctctcgccatgaagcagctcaatctgaatgacaacgctattcacctttatgcctctgtcttctccaacaacgctcgaaggctccactctgacgcctctaagaataaggagaaggctctgatcattatcaagagtctgctttcgccgtatggcaaaacaaatctgacggacgccctgcttcaggtccggaagcacttgaacgaccggaagaatgccaatcagttggtcgtgatcctc

SEQ ID NO: 4 - DNA, 296 bp, Homo sapiens
aacaaaaacaatcagggaaatggccaaggtcacaatgacccaaatagaaacgtagacgagaatgccaatgctaactctgctgtaaagaacaacaataacgaagacaagcatattaaagaatatttgaacaaaattcagaatagcttgtccacagagtggtcaccatcgtgcgggaacgggatccaagtccggatcaaacctggaagcgccaataaacccaaggacgagcttaacgatatcgagaagaaaatatgtaaaatggagaagtgtagctccgtgtttaacgtagtcaatag